(19) United States
(12) Patent Application Publication
(10) Pub. No.: US 2005/0059894 A1
(43) Pub. Date: Mar. 17, 2005

(54) Automated endoscopy device, diagnostic method, and uses

(76) Inventors: Haishan Zeng, Vancouver (CA); Mirjan Petek, Richmond (CA); James Dao, Henderson, NV (US); Branko Palcic, Vancouver (CA); Gary Ferguson, Burnaby (CA)

Correspondence Address: TREXLER, BUSHNELL, GIANGIORGI,;BLACKSTONE & MARR, LTD., 105 WEST ADAMS STREET, SUITE 3600, CHICAGO, IL, 60603, US

(21) Appl. No.: 10/663,998

(22) Filed: Sep. 16, 2003

(51) Int. Cl. A61B 6/00

(52) U.S. Cl. 600/476; 600/160

(57) ABSTRACT

The present invention is an automated endoscopic device and diagnostic method, which performs at least one other disease detection method simultaneously during a white light endoscopic procedure. In some embodiments fluorescence imaging or spectroscopy is performed during the white light examination. In other embodiments, multi-modal imaging and/or spectroscopy may be performed and combined in a variety of ways. Because diagnostic modes other than white light are performed transparently in the background, the procedure is not significantly more complex for the clinician than the familiar white light examination. In some embodiments the present invention automatically detects suspicious tissue and informs the clinician of its presence. In other embodiments the present invention helps determine if a biopsy is required, and may further assist the clinician, for example, by providing an outline or otherwise guide the clinician in identifying and/or taking a biopsy of a suspicious site. In yet other embodiments, the present invention includes refinements afforded by incorporating a priori information, for example, patient history, previous endoscopy data, the results of qualitative and/or quantitative sputum cytology etc.

### BACKGROUND OF THE INVENTION

Imaging is capturing electromagnetic radiation (wavelength and intensity) either reflected or emitted from an object of interest, in a manner which preserves or otherwise represents the spatial distribution of said radiation at the object. In the field of medical imaging, and more particularly endoscopy, light is utilized to illuminate body tissues and return a diagnostic or otherwise useful image. Historically, clinicians viewed white light reflectance (color) images through an ocular attached to the endoscope. More recently, with cost reductions and other computer advances, rather than viewing a tissue image through an ocular, endoscopic images are typically displayed on a monitor. Bronchoscopy serves as an example of a specific endoscopic procedure, in this instance for examining the lungs and respiratory tract. When white light is used for tissue illumination it provides visual indication of the physical structure (morphological image) of the lungs and bronchial passages. In use, clinicians may detect various diseases such as lung cancer by observing features in white light reflectance images such as the color and surface morphology of lung tissue and its various structures.

White light means a broad spectrum or combination of spectra in the visible range. For endoscopy, typically LEDs, lamps, lasers alone or in combination, along with optical elements such as lens, filters, filter wheels, liquid-crystal filters and multi-mirror devices, are used to provide the desired white-light illumination. It is considered advantageous for the clinician to be presented with a white-light image in real-time (at video rate). At the same time as images are displayed, images may be captured and analyzed by computer to extract various features. Accordingly, it is an object of the present invention to provide a white-light image to guide or otherwise utilize an endoscope. It is a further object of the present invention to analyze white-light images and utilize this information to automate the endoscopic device, as will be discussed further herein. It is yet a further object of the present invention in various embodiments, to perform visible reflectance spectroscopy in real-time and to utilize these spectral measurements to further automate the device.

Medical research indicates that cancer may be treated more effectively when detected early when lesions are smaller or when tissue is in a precancerous stage. While changes in the physical appearance (color and morphology) of tissue using white light is useful, to accomplish more reliable and earlier detection of diseases, such as cancer, various endoscopic imaging devices have been developed which have increased sensitivity to the biological composition of tissue. Just as certain morphological changes in tissue may be associated with disease, chemical changes may also be exploited for disease detection.

One such method of detecting chemical changes in tissue during an endoscopic procedure involves utilizing tissue illumination with specific wavelengths or bands of light that interact with certain chemical compounds in tissue, particularly those that are associated with diseases, such as cancer. For example, some endoscopic devices utilize light in the UV or UV/blue spectrum to illuminate tissue. These wavelengths of light are selected based on their ability to stimulate certain chemicals in tissue that are associated with disease, or disease processes.

For example, when illuminated with UV or UV/blue light, tissue may emit light at wavelengths longer than the illumination (also called excitation) light and images or spectra from these tissue emissions (fluorescence) may be captured for observation and/or analysis. Healthy and diseased tissue fluoresce differently, so the spectra of fluorescence emissions can be used as a diagnostic tool.

In addition, to assist in interpreting these fluorescence images, pseudo-colors may be assigned to help visualize the extent and location of diseased tissue. For example, the color red may be assigned to diseased tissue while healthy tissue may be displayed in green. As with any subjective method, standardization becomes problematic and establishing particular color tones or intensities as well as matching image characteristics from instrument to instrument or between devices available from different manufacturers may complicate matters.

“Spectroscopy” here refers to the analysis of light according to its wavelength or frequency components. The analysis results are usually presented in the form of spectrum or spectra, which is a plot of light intensity as a function of wavelength. Reflectance spectroscopy is the analysis of reflected light from the tissue. Biological tissue is a turbid medium, which absorbs and scatters incident light. The majority of the reflected light from tissue has traveled inside the tissue and encountered absorption and scattering events, and therefore contains compositional and structural information of the tissue.

Tissue reflectance spectroscopy can be used to derive information about tissue chromophores (molecules that absorbs light strongly), e.g. hemoglobin. The ratio of oxyhemoglobin and deoxy-hemoglobin can be inferred and used to determine tissue oxygenation status, which is very useful for cancer detection and prognosis analysis. It can also be used to derive information about scatterers in the tissue such as the size distribution of cell nucleus and average cell density.

Fluorescence spectroscopy is the analysis of fluorescence emission from tissue. Native tissue fluorophores (molecules that emit fluorescence when excited by appropriate wavelengths of light) include tyrosine, tryptophan, collagen, elastin, flavins, porphyrins, and nicotinamide adenine dinucleotide (NAD). Tissue fluorescence is very sensitive to chemical composition and chemical environment changes associated with disease transformation. Fluorescence imaging takes advantage of fluorescence intensity changes in one or more broad wavelength bands thus providing sensitive detection of suspicious tissue areas, while fluorescence spectroscopy (especially spectral shape) can be used to improve the specificity for early cancer detection.

Although fluorescence (imaging) endoscopy provides increased sensitivity to diseases such as cancer, there are also some trade offs. For example, while sensitivity is increased (something abnormal is indicated), specificity is reduced, causing some non-diseased tissue (e.g. benign tissue) to mimic the chemical signatures of diseased tissue (e.g. cancer), thus making the colored images indistinguishable from true disease. These additional suspect tissue sites (false positives) may require further investigation to confirm disease status; for example, the clinician may need to take a biopsy for examination by a pathologist. Another limitation of fluorescence imaging endoscopy is that it does not provide the same image quality for morphological structure and therefore typically requires additional caution, and time to guide the endoscope during the procedure. In addition, of those clinicians capable of performing white-light endoscopy, only a small percentage of them are experienced and proficient at performing fluorescence endoscopy. It is therefore an object of the present invention to perform fluorescence imaging, fluorescence spectroscopy, and reflectance spectroscopy as background tasks, simultaneously with white-light imaging/assessment.

Endoscopic devices are available which perform both white light and fluorescence imaging. Some of these systems provide various imaging modalities (white light imaging and fluorescence imaging) in sequence whereas other devices perform both imaging modes, simultaneously. Co-pending United States patent application to Zeng, entitled, “Real time contemporaneous multi-modal imaging and spectroscopy uses thereof”, and co-pending United States patent application to Zeng, entitled, “Methods and apparatus for fluorescence imaging and multiple excitation-emission pairs and simultaneous multi-channel image detection” as well as co-pending United States patent application to Zeng, entitled, “Methods and apparatus for fluorescence and reflectance imaging and spectroscopy and for contemporaneous measurements of electromagnetic radiation with multiple measuring devices”, describe various hardware configurations and methods for simultaneous multi-modal imaging and detection, appropriate for exploitation by the present invention.

While some advances facilitate the endoscopy procedure, they do not fully address the issue of lost specificity, which typically results when the more disease-sensitive fluorescence imaging modality comprises part of the procedure.

In view of these endoscopic developments and limitations, it is an object of the present invention to provide endoscopic devices and methods which mimic the familiar white-light endoscopy procedure but which integrate other detection modalities in a manner that is relatively transparent (performed as a background task) to the clinician, therefore providing an improvement in comfort and efficiency. In addition, the present invention may also provide a means to recover some of the specificity that is lost during fluorescence endoscopy by combining detection modalities, such as spectroscopy. Accordingly, embodiments of the present invention may provide the clinician with a white-light image, while fluorescence and other assessments (e.g. fluorescence imaging, fluorescence spectroscopy, reflectance spectroscopy, image analysis etc.) occur transparently in the background. It is a further object of the present invention to automatically detect suspicious tissue and inform the clinician that disease may be present. It is yet another object of the present invention to indicate (e.g. by outlining an image region), to further assist the clinician in taking a biopsy. And it is yet a further object of the present invention to help determine if a biopsy is required, for example by including a priori information, such as patient history, subjective and/or objective cytology, tissue spectroscopy, etc. during the procedure.

### Discussion of Related Art

U.S. Pat. No. 6,061,591, to Freitag, entitled, “Arrangement and method for diagnosing malignant tissue by fluorescence observation”, discusses switching between white light and fluorescence visualization methods.

U.S. Pat. No. 5,647,368, to Zeng, entitled, “Imaging system for detecting diseased tissue using native fluorescence in the gastrointestinal and respiratory tract”, among other things discusses use of a mercury arc lamp to provide for white light and fluorescence imaging with an endoscope to detect and differentiate normal from abnormal or diseased tissue.

U.S. Pat. No. 5,590,660, to MacAulay, entitled, “Apparatus and method for imaging diseased tissue using integrated autofluorescence” discusses light source requirements, optical sensors, and means to provide a background image to normalize the autofluorescence image, for uses such as imaging diseased tissue.

U.S. Pat. No. 5,769,792, to Palcic, entitled, “Endoscopic imaging system for diseased tissue”, further discusses light sources and means to extract information from the spectral intensity bands of autofluorescence, which differ in normal and diseased tissue.

Also co-pending U.S. patent application Ser. No. 09/741,731 to Zeng, entitled “Methods and apparatus for fluorescence and reflectance imaging and spectroscopy and for contemporaneous measurements of electromagnetic radiation with multiple measuring devices”, and its continuation-in-part of the same title, application Ser. No. 10/028,568, U.S. Publication No. 2002/0103439, discuss contemporaneous methods of imaging and spectroscopy.

U.S. Pat. No. 6,212,425 to Irion, entitled “Apparatus for photodynamic diagnosis”, discusses endoscopic imaging using a light-induced reaction or intrinsic fluorescence to detect diseased tissue and delivery light for therapeutic use or to stimulate compounds that in turn provide therapy, for example.

Endoscopes and imaging applications are further discussed in co-pending U.S. patent application Ser. No. 10/226,406 to Ferguson/Zeng, entitled “Non-coherentfiber optic apparatus and imaging methods”, which, among other things, discusses apparatus to overcome some existing limitations of fiber optic devices, such as endoscopes.

Co-pending U.S. patent application Ser. No. 10/431,939 to Zeng, entitled “Real-time contemporaneous multimodal imaging and spectroscopy and uses thereof”, among other things, discusses various devices and configurations for simultaneous white light and fluorescence imaging.

Co-pending U.S. patent application Ser. No. 10/453,040 to Zeng, entitled “Methods and apparatus for fluorescence imaging and using multiple excitation-emission pairs and simultaneous multi-channel image detection” among other things discusses means to excite and image more than one fluorescence channel, alone or in combination with white light imaging.

U.S. Pat. No. 6,366,800 to Vining, entitled “Automatic analysis of virtual endoscopy”, among other things, discusses computer analysis, construction of three dimensional images from a series of two dimensional images, and using wire frame models to represent data to indicate, for example, abnormal wall structure.

U.S. Pat. No. 6,556,696 to Summers, entitled “Method of segmenting medical images and detecting surface anomalies in anatomical structures”, among other things, discusses computer analysis and decision making using neighboring vertices, curvature characteristics and other factors as well as computing the position of a lesion and forming desired composite images for display.

### BRIEF SUMMARY OF THE INVENTION

The present invention is an automated endoscopic platform/device and diagnostic method, which performs at least one other disease detection method, such as reflectance imaging, fluorescence imaging, spectroscopy etc. simultaneously as a background task during a white light endoscopic procedure. In one embodiment, the apparatus and method involve using white light to guide the endoscope, while fluorescence images are collected and analyzed. If suspect tissue is detected, the user is alerted. In another embodiment, if suspect tissue is detected, the area of that tissue is delineated or highlighted for display and a spectroscopic analysis is initiated. In another embodiment, prior information such as risk factors or other laboratory tests is combined with the results of the fluorescence imaging and/or spectroscopic analysis to determine if a biopsy or other procedure is indicated. In another embodiment, a third-party plug in analyzer is used simultaneously in the endoscope, and the results of that plug-in analysis are combined with the date generated as described above to determine what further action is needed. In all of the above embodiments, any combination of the results of the various imaging and spectrographic analysis and the prior information can be combined to yield a quantitative score, which can be compared to a benchmark score stored in a database to determine if biopsy or other procedure is indicated.

This platform/device also allows the integration of a third-party endoscopy positioning system (EPS) to guide the advancement and maneuver of the endoscope inside the body cavities. The system software also facilitates the annotation and marking of a detected suspicious area in the EPS mapping system (or EPS map) and facilitates convenient re-visit of the suspicious site for further diagnostic analysis, therapy and follow-up. When revisit a marked site, all previously stored information (images, spectra, quantitative scores etc.) can be recalled and displayed on the monitor for the attending physician's reference.

### BRIEF DESCRIPTION OF THE SEVERAL VIEWS OF THE DRAWINGS

FIG. 1 shows a basic embodiment of the present method.

FIG. 2 shows another embodiment of the present method incorporating spectroscopy.

FIG. 3 shows the present invention utilizing a priori data within the diagnostic method.

FIG. 3b shows the method of FIG. 3 with addition of plug-in analysis.

FIG. 3c shows the method of FIGS. 3 and 3b with addition of annotation of the suspicious site on EPS map.

FIG. 4 shows a white light image display with lesion boundaries delineated by background fluorescence imaging analysis.

FIG. 5 shows a hardware embodiment of the present device with spectroscopy.

FIG. 6 shows another hardware embodiment for simultaneous multi-modal imaging and spectroscopy.

FIG. 7 shows a spectroscopy configuration.

FIG. 8 shows another configuration for spectroscopy.

FIG. 9a shows a simple configuration of the present invention

FIG. 9b shows various display options and features for the present invention

### DETAILED DESCRIPTION OF THE INVENTION

The organization and manner of the preferred embodiments of the invention, together with further objects and advantages thereof, may best be understood by reference to the following description, taken in connection with the following drawings:

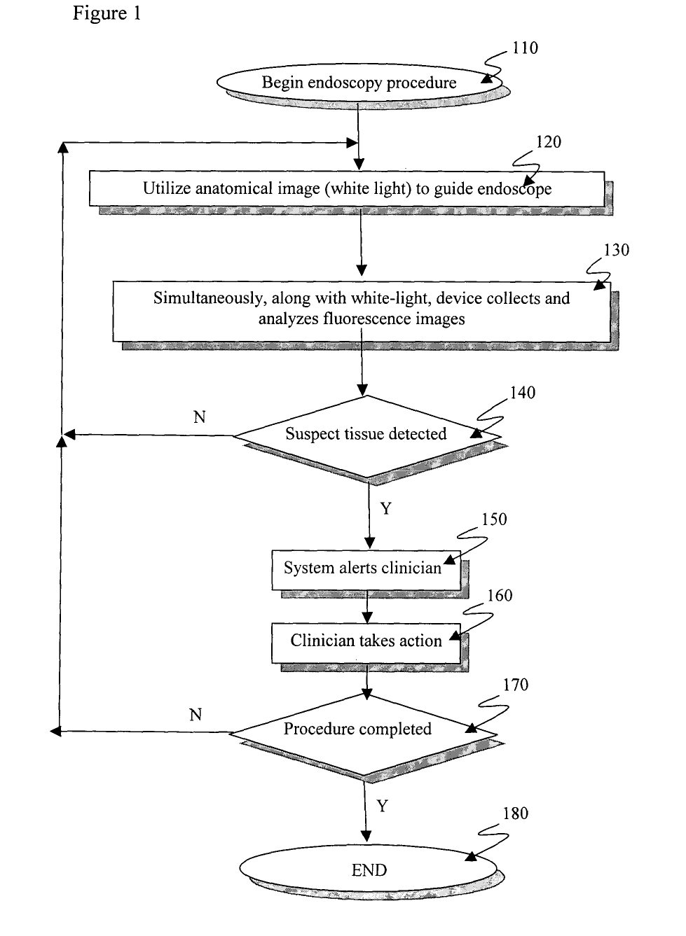

FIG. 1 shows a basic embodiment of the present invention with automated endoscopy method beginning at 110. The clinician is provided with an anatomical image 120 comprised of one or more bands of light, which carry sufficient spectral content to render gross morphology, visible. Typically such an anatomical image is formed from relatively broad-band reflected light, however, such an image may also be formed from combining various spectra and as required or desired may also include fluorescence components. Utilizing this white light, or comparable image for guiding the endoscope, the device simultaneously collects and analyzes fluorescence images 130. While white light may provide some useful information, fluorescence imaging provides improved detection for some diseases, such as cancer. In the event that suspect tissue is detected 140 by fluorescence imaging, the device alerts the clinician 150, audibly or visibly. The clinician may then take various steps 160, for example, the clinician may manually switch the device to display fluorescence images, or the device may be enabled to automatically display fluorescence or other composite images when a suspected abnormality is detected. In addition, software may provide support indicators, such as highlighting or drawing boundaries around the suspect tissue site. Such information and guidance may be useful in detecting disease and further assisting the clinician by guiding a biopsy, treatment, tissue excision or other step in the diagnosis or management of the disease. The procedure continues 170 or ends 180 when complete.

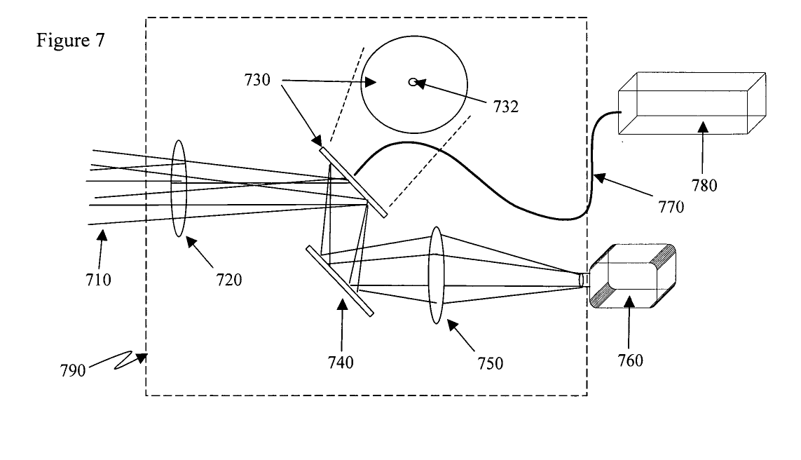

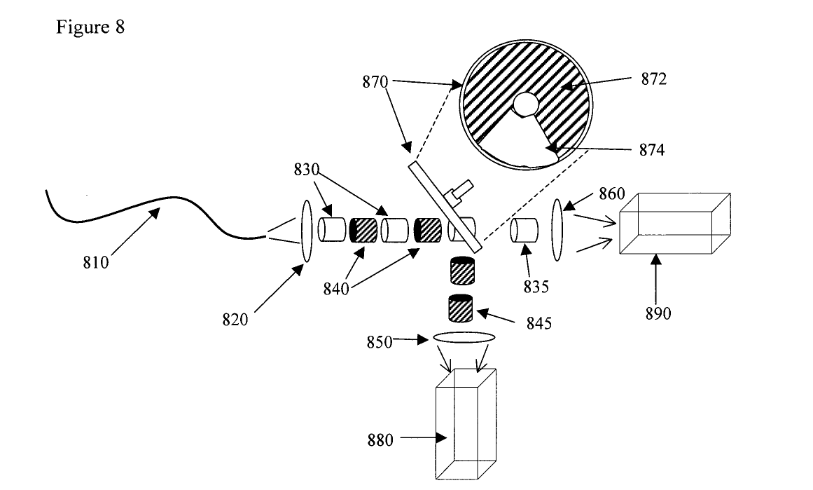

During the endoscopic procedure, spectroscopy (reflectance and/or fluorescence) or image analysis may be performed in real-time and this information may be used in various ways to provide a more automated endscopic device, as contemplated herein. For example, the results of the spectroscopic or image analysis can be assigned a quantitative score. This score can be compared to benchmark scores stored in a database to determine if further procedures, such as surgery or biopsy, are required. Spectroscopy configurations are further discussed in association with FIGS. 7 and 8, herein. In this manner, a more sensitive, multi-modal endoscopy examination may be accomplished which to the clinician, closely resembles the familiar white-light endoscopy procedure.

Real-time image analysis here refers to image analysis operations performed within a few milliseconds (ms) so that images can be acquired, processed, and displayed in real time (or video rate, 30 frames/sec). For example, images from different channels can be mirror flipped in real time for alignment purposes. Images from different channels can also be shifted pixel by pixel along X-Y directions in real time again for the alignment of images from different channels. The ratios of the green channel image to the red channel image of a fluorescence image can be calculated pixel by pixel in real time to form a new image. A threshold detection procedure could then be applied to this image to segment out the suspicious diseased area based on the fact that cancerous lesions typically have lower green/red ratios. These tasks can be performed in real time to render a line, highlight or other boundary/indicator on the white light image as a visual aid to delineate the lesion.

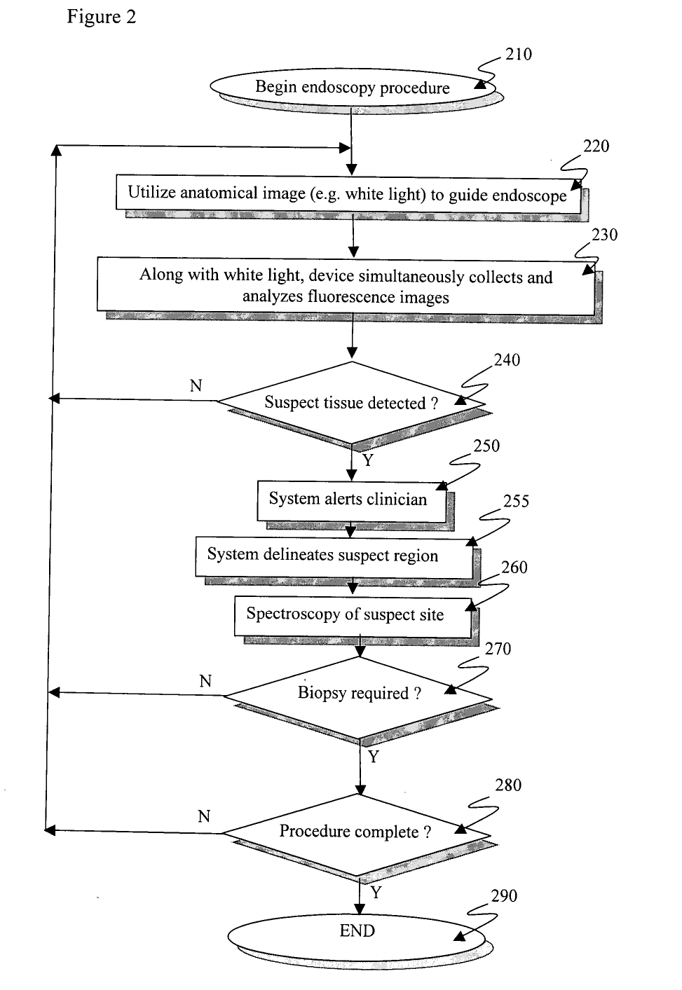

FIG. 2 shows another embodiment of the present invention with automated endoscopy method beginning at 210. The clinician is provided with an anatomical image 220 comprised of sufficient spectral content to render gross morphology, visible. Utilizing this image to guide the endoscope, the device simultaneously collects and analyzes fluorescence images 230. Although white light may provide some useful information for detecting disease such as redness or inflammation, fluorescence imaging provides improved sensitivity for some diseases, such as cancer. In the event that suspect tissue is detected 240 by fluorescence imaging, the device alerts the clinician 250 who may then take various steps. Accordingly, in support, the device (manually or automatically) may be activated to display various useful images, for example, fluorescence or composite images. Such composite images may include highlighting, boundaries or other indicators that help delineate the suspect tissue region 255. Combined information or composite images 255 may support other diagnostic steps, for example, targeting spectroscopy 260 to further assess the suspect tissue to further indicate if a biopsy 270 is required. The procedure proceeds 280, until complete 290.

Endoscopy may be used as illustrated to detect disease or may be used in follow-up or as part of a treatment protocol.

Accordingly, the present invention may provide a high sensitivity, multi-modal examination, which more closely resembles the familiar white-light endoscopy procedure. The issues of sensitivity, specificity, simultaneous white light and fluorescence as well as invoking spectroscopy as a means to better determine whether a biopsy is required are discussed in co-pending patent applications to Zeng. One of these is U.S. patent application Ser. No. 10/431,939 entitled “Real-time contemporaneous multimodal imaging and spectroscopy uses thereof”, which, among other things, discusses various devices and configurations for simultaneous white light and fluorescence imaging. Also, U.S. patent application Ser. No. 10/453,040 to Zeng entitled “Methods and apparatus for fluorescence imaging and multiple excitation-emission pairs and simultaneous multi-channel image detection” among other things discusses means to excite and image more than one fluorescence channel, alone or in combination with white-light imaging.

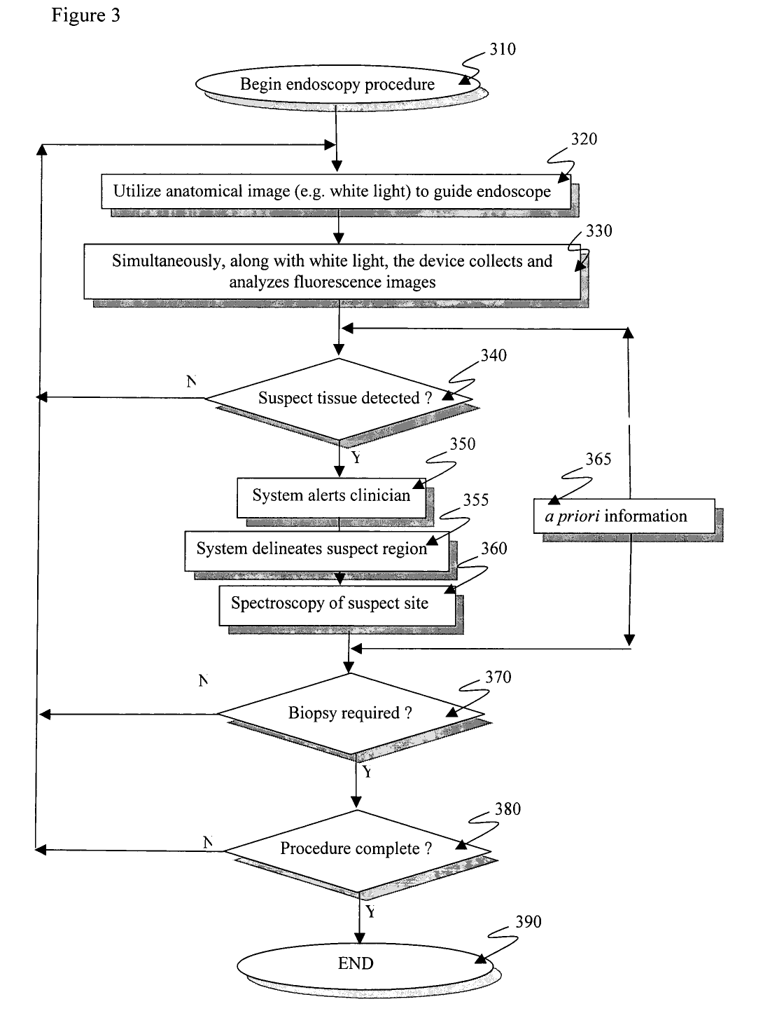

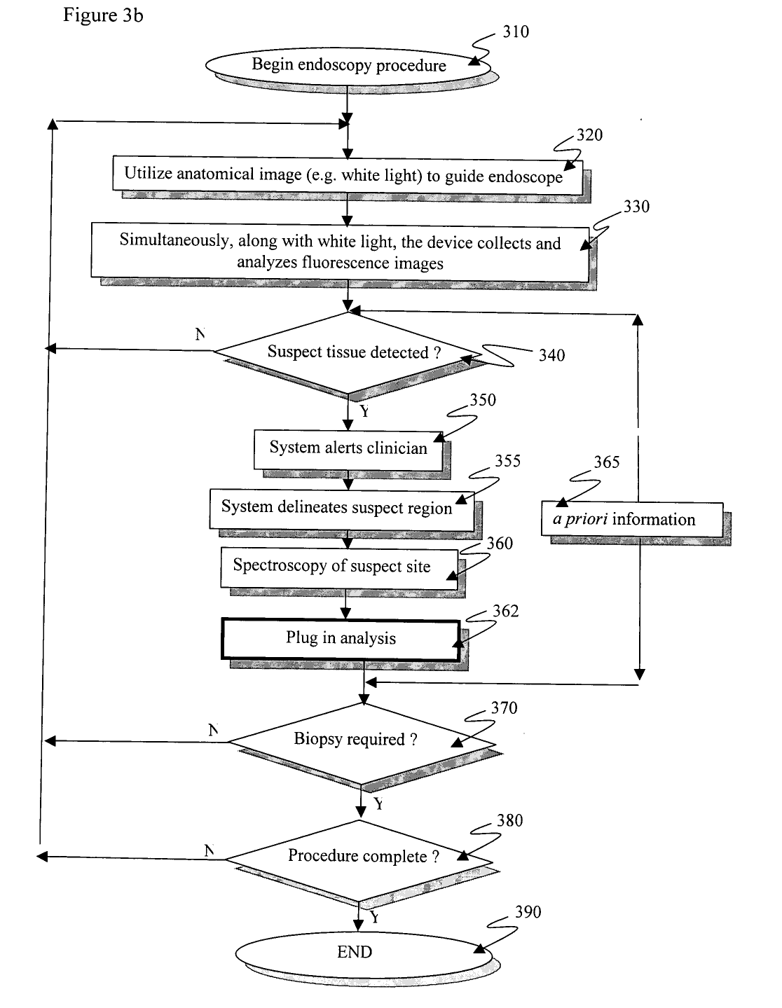

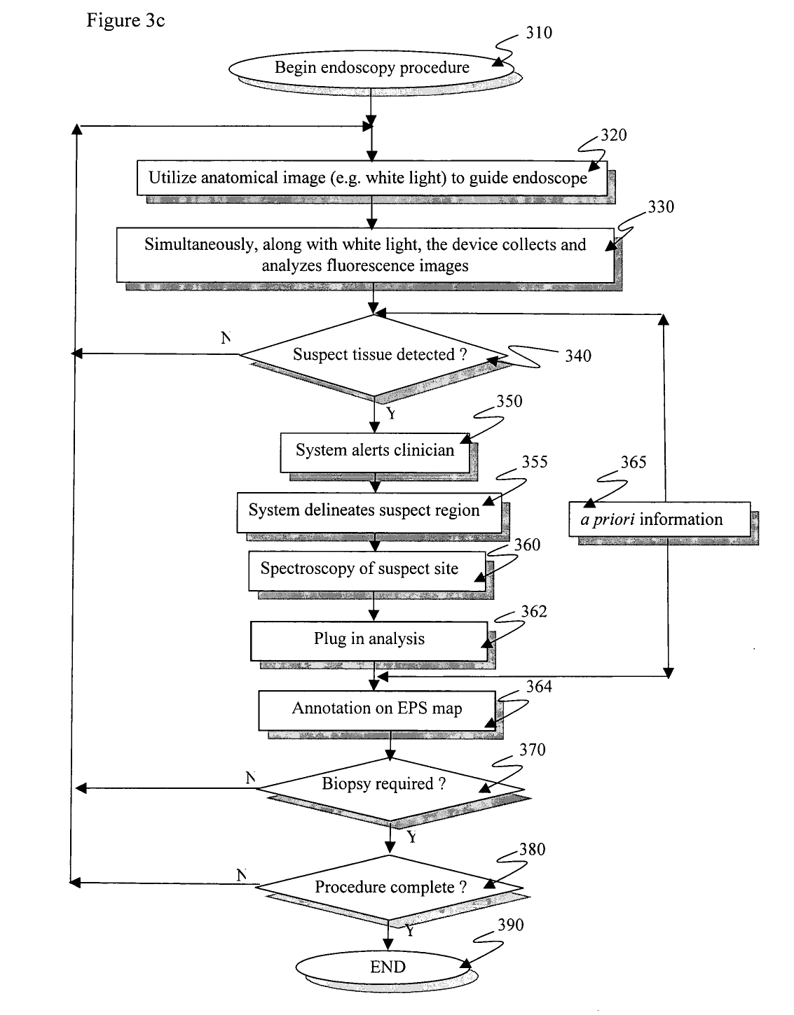

FIG. 3a illustrates another embodiment of the present invention with automated endoscopy method beginning at 310. The clinician is provided with an anatomical image 320 comprised of sufficient spectral content to render gross morphology, visible. Utilizing this image to guide the endoscope, the device simultaneously collects and analyzes fluorescence images 330. In the event that suspect tissue is detected 340 by the device based upon analysis of white light and/or fluorescence images or other factors 365 to be further discussed, the device alerts the clinician 350 who may then take various steps. In support of these decisions, the device may manually or automatically change display modes; for example, at 355 boundaries determined from the analysis of fluorescence images may be displayed onto a white light image. Spectroscopy 360 may then be performed on the suspect tissue either automatically or be directed interactively by the clinician. Such spectroscopy information may help determine the extent of disease, treatment or better indicate 370 whether a biopsy is required.

Various a priori information 365 may be used to adjust decisions nodes. For example this a priori information may include risk factors, smoking history, patient age, x-ray or other imaging data, or diagnostic test results such as, for example, blood chemistry, antibody or genetic marker status, or qualitative and/or quantitative cytology of sputum or other tissue samples. The results of the spectroscopic or image analysis can be combined with this prior information and assigned a quantitative score. This score can be compared to benchmark scores stored in a database to determine if further procedures, such as surgery or biopsy, are required. The procedure continues 380 until complete 390.

FIG. 3b illustrates another embodiment of the present invention with automated endoscopy method beginning at 310. As in FIG. 3a, the clinician is provided with an anatomical image 320 comprised of sufficient spectral content to render gross morphology, visible. Utilizing this image to guide the endoscope, the device simultaneously collects and analyzes fluorescence images 330. In the event that suspect tissue is detected 340 by the device based upon analysis of white light and/or fluorescence images or other factors 365 to be further discussed, the device alerts the clinician 350 who may then take various steps. In support of these decisions, the device may manually or automatically change display modes; for example, at 355 boundaries determined from the analysis of fluorescence images may be displayed onto a white light image. Spectroscopy 360 may then be performed on the suspect tissue either automatically or be directed interactively by the clinician. Such spectroscopy information may help determine the extent of disease, treatment or better indicate 370 whether a biopsy is required.

Apart from reflectance and fluorescence spectroscopic analysis with the build-in devices of the system, the system also serves as a basic endoscopy platform, utilizing third-party plug-in analysis 362 to support use of various catheters and probes introduced through the instrument channel of the endoscope. These plug-in analyses will further help the clinician with decision making. For example, a Raman probe/catheter as illustrated in U.S. Pat. No. 6,486,948 to Zeng entitled “Apparatus and Methods Related to High Speed Raman Spectroscopy” and in co-pending U.S. Provisional Patent Application No. 60/441,566 by Zeng, entitled “Raman Endoscopic Probe and Methods of Use”, can be introduced to acquire Raman spectra from the diseased site to further improve the detection specificity and provide information on changes of protein contents and genetic materials in cancerous lesions that will help in predicting the malignancy potential and the prognosis of the lesion. Raman spectroscopy can also be used to monitor drug delivery and treatment effectiveness during therapy.

Another plug-in spectroscopy analysis could be fluorescence excitation-emission matrix (EEM) spectroscopy as illustrated in U.S. Provisional Patent Application No. 60/425,827 by Zeng et al., entitled “Apparatus and methods related to high speedfluorescence excitation-emission matrix (EEM) spectroscopy”. The EEM analysis will further improve the detection specificity and help with predicting the prognosis of the lesion.

Another example of plug-in analysis is Optical Coherence Tomography (OCT) and confo cal microscopy as illustrated in U.S. Pat. No. 6,546,272 to MacKinnon et al., entitled “Apparatus for in vivo imaging of the respiratory tract and other internal organs”, and U.S. Pat. No. 20,030,076,571A1 to MacAulay entitled “Methods and apparatus for imaging using a light guide bundle and a spatial light modulator.” OCT and confocal microscopy allow depth profiling of tissue sites of interest and can be used to determine the depth of the lesion (invasiveness of dysplasia or tumor) that will assist in biopsy procedure and therapy. A pathologist may be connected by Internet to view these sectional images during the endoscopy procedure and provide their opinion regarding the necessary of biopsy or perform diagnosis online and invoke immediate decision regarding therapy.

Various a priori information 365 may be used to adjust decisions nodes. This a priori information may include risk factors, smoking history, patient age, x-ray or other imaging data, diagnostic test results such as blood chemistry, antibody or genetic marker status, or qualitative and/or quantitative cytology, for example. The results of the spectroscopic or image analysis can be combined with the prior information and/or with the results of the plug-in analyzer and be assigned a quantitative score. This score can be compared to benchmark scores stored in a database to determine if further procedures, such as surgery or biopsy, are required. The procedure continues 380 until complete 390.

FIG. 3c illustrates another embodiment of the present invention with automated endoscopy method beginning at 310. As in FIG. 3b, the clinician is provided with an anatomical image 320 comprised of sufficient spectral content to render gross morphology, visible. Utilizing this image and a third-party EPS integrated with the present system to guide the endoscope, the device simultaneously collects and analyzes fluorescence images 330. In the event that suspect tissue is detected 340 by the device based upon analysis of white light and/or fluorescence images or other factors 365 to be further discussed, the device alerts the clinician 350 who may then take various steps. In support of these decisions, the device may manually or automatically change display modes; for example, at 355 boundaries determined from the analysis of fluorescence images may be displayed onto a white light image. Spectroscopy 360 may then be performed on the suspect tissue either automatically or be directed interactively by the clinician. Such spectroscopy information may help determine the extent of disease, treatment or better indicate 370 whether a biopsy is required.

Apart from reflectance and fluorescence spectroscopic analysis with the build-in devices of the system, the system also serves as a basic endoscopy platform, utilizing third-party plug-in analysis 362 to support use of various catheters and probes introduced through the instrument channel of the endoscope. These plug-in analyses will further help the clinician with decision making.

Various a priori information 365 may be used to adjust decisions nodes. This a priori information may include risk factors, smoking history, patient age, x-ray or other imaging data, diagnostic test results such as blood chemistry, antibody or genetic marker status, or qualitative and/or quantitative cytology, for example. The results of the spectroscopic or image analysis can be combined with the prior information and/or with the results of the plug-in analyzer and be assigned a quantitative score. This score can be compared to benchmark scores stored in a database to determine if further procedures, such as surgery or biopsy, are required.

The suspicious site can be annotated on the EPS map in step 364 along with storing of all the images, spectra, third-party plug-in analysis output, online pathologist's input, and the prior information for this site. This annotation or marking will facilitate convenient revisit of the site for follow-up and/or therapy purposes. All the stored data and information related to this site can be recalled for reference during the re-visit. The procedure continues 380 until complete 390.

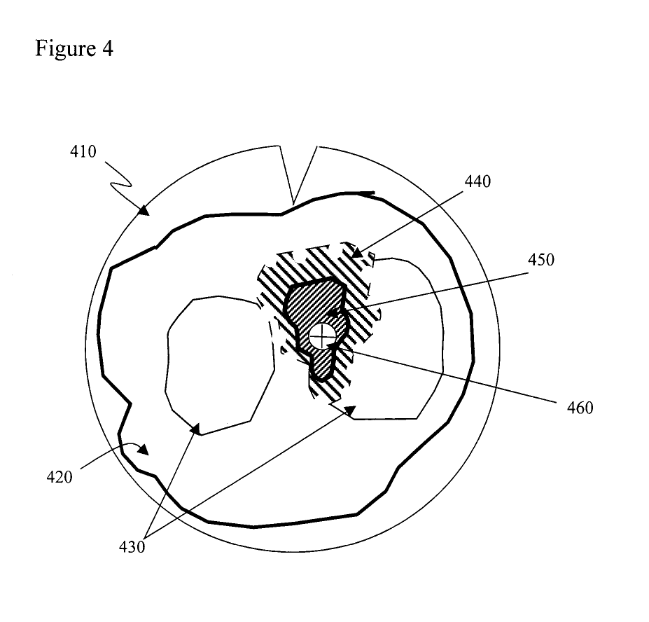

FIG. 4 further describes various steps in an automated endoscopy procedure. In this instance, endoscopic lung image 410 provides an anatomical view of lung tissue 420 having bronchial passages 430 and suspect tissue lesion 440 with irregular boundary detected by analysis of fluorescence images. Once a suspect site is detected, a variety of images may be usefully displayed separately on the monitor in combined form. In this example, a portion of the fluorescence image indicative of diseased tissue is displayed overtop the anatomical white light image. In addition, computer image analysis has performed a fluorescence intensity profile, providing information to identify more accurately the suspect tissue site 450. Subsequently within area 450, spectroscopy 460 may be guided to help determine, for example, if a biopsy of the suspect tissue site is required.

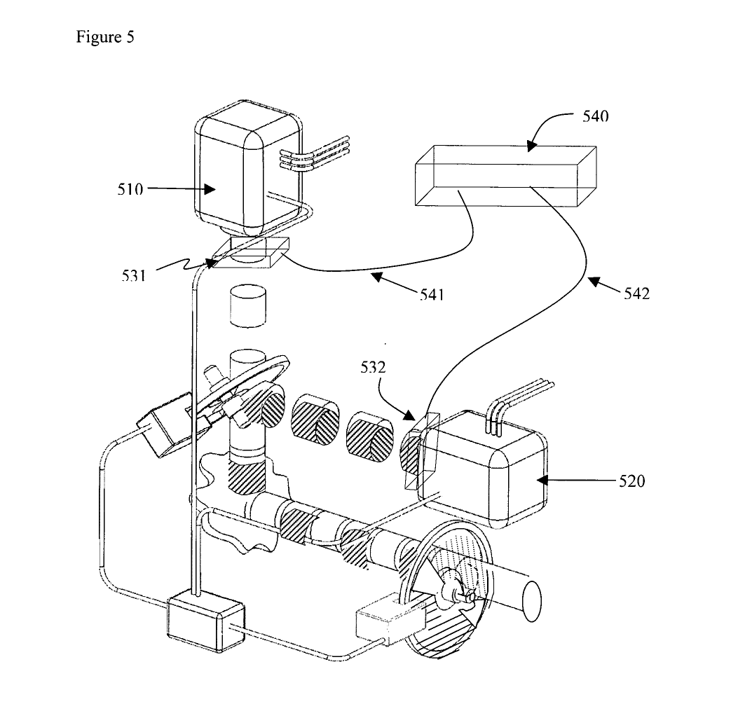

FIG. 5 shows an endoscopy device capable of simultaneous real-time white light and fluorescence imaging such as described in co-pending applications to Zeng referenced above. In this instance, the system has both a white-light imaging detector 510 and a fluorescence imaging detector 520. Corresponding spectral attachments 531 and 532 have connecting optical fibers 541 and 542 which provide for spectroscopy at desired times on suspect tissue, for example, when suspicious tissue identified by visual abnormalities within the white light image or by fluorescence imaging. Accordingly, dual channel, or multiplexed spectrometer 540 provides for spectral measurements as required, or desired.

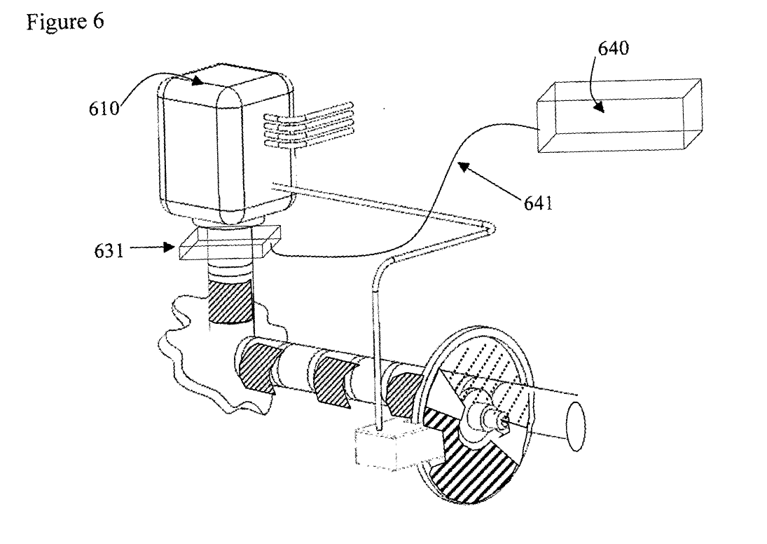

FIG. 6 shows another endoscopy device providing contemporaneous white light and fluorescence imaging, in this instance, utilizing a single detector 610, which contains multiple sensors to accomplish multi-modal imaging. Such devices and optical configurations are described in co-pending United States patent applications to Zeng as referenced above. A spectral attachment 631 routes photons containing spectral information via fiber 641 to a spectrometer 640. These spectra may be used, for example, to assessing suspect tissue to help determine whether a biopsy is required.

FIG. 7 illustrates means of providing simultaneous endoscopic imaging with spectral information, including white light and fluorescence information 710 focused by lens 720 onto a fiber mirror 730. The vast majority of this image is directed to mirror 740 and the image focused by lens 750 for capture by imaging detector 760. A fraction of the image is captured via an optical fiber 770 through a small orifice 732 formed in the fiber mirror 730. Fiber mirror 730 is further shown in projected view with the orifice 732 providing means for the optical fiber to receive spectral information which is further directed to spectrometer 780. The boxed area 790 further indicates the location of spectroscopy components associated with FIG. 5 (531, 532) and FIG. 6 (631).

FIG. 8 shows the details of spectrometer 640 with light containing spectral content carried by optical fiber 810 and collimated by lens 820. Typically for real-time multi-modal imaging, segments of white light and fluorescence content arrive at video rate. These alternating white-light segments are further indicated as 830 and fluorescence light segments as 840. As illustrated these light segments then interact with rotating filter wheel 870, which is further shown to have reflective region 874 and light passing/processing filter region 872. The filter region 872 may be further comprised of multiple filter regions to process spectral components, for example to separate red, blue and green light. Processed white light segments such as 835 proceed to lens 860 and are directed to spectrometer 890. Fluorescence light segments 840 are reflected by region 874 of rotating filter wheel 870 and these reflected light segments 845 are focused by lens 850 onto spectrometer 880. As required or desired since the spectral packages of white light and fluorescence light are already separated in time domain, they may also be multiplexed to a single spectrometer.

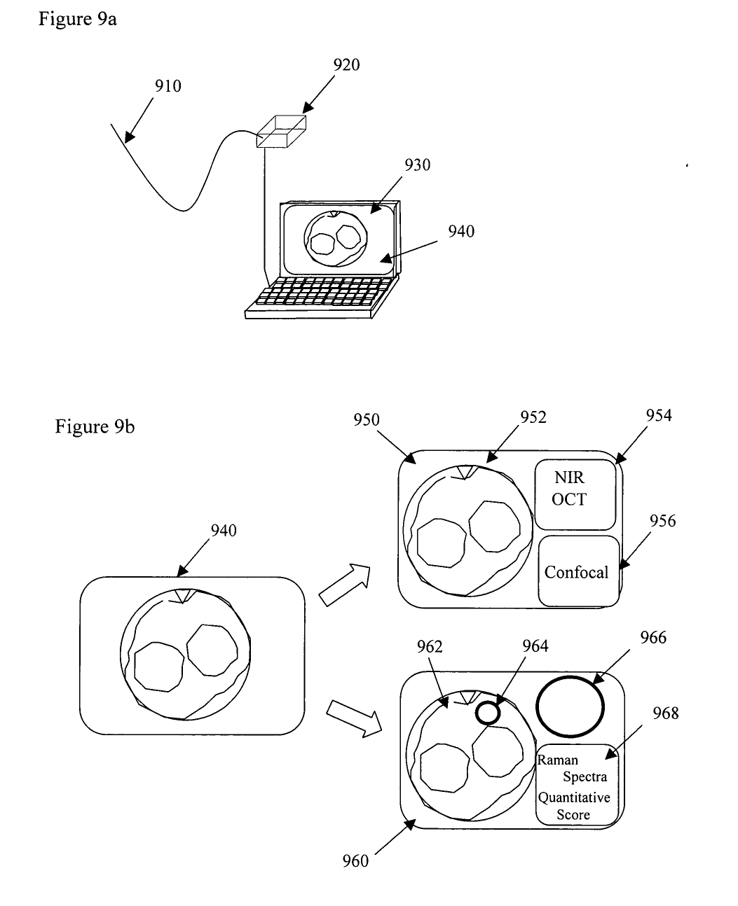

FIG. 9a shows a simple, low cost configuration of the present invention comprised of endoscope 910 providing real-time, multi-modal images such as white light and fluorescence to imaging camera 920. Images are captured, analyzed and displayed by a computer/monitor such as laptop computer 930. For basic operation the primary image displayed is white light image 940.

FIG. 9b shows white light image 940 used to guide an endoscopic procedure. Subsequent to computer image analysis detecting a suspicious tissue region, the display switches to a pallet of diagnostic images/data 950, 960. Further represented in image 950 are the white light image 952, images/data derived from optical computer tomography and near infrared fluorescence imaging 954 as well as in this instance, confocal microscopy images/data 956. Similarly, composite image 960 illustrates a white light image 962 with highlighted suspect region 964. The suspect regions is further enlarged 966 while spectral and quantitative data (a priori information) 968 are displayed to further assist the clinician, for example to deduce whether a biopsy of the suspicious region is required or desired.

While preferred embodiments of the present invention are shown and described, it is envisioned that those skilled in the art may device modifications of the present invention without departing from the spirit and scope of the appended claims.

1. An automated device for imaging and diagnosis of a target, comprising:
an endoscope,
a first means for performing a white-light assessment of the target, and
a second means for performing an additional assessment of the target as a background task.

2. The device of claim 1, wherein said additional assessment comprises at least one fluorescence imaging mode.

3. The device of claim 1, wherein said additional assessment comprises at least one of reflectance spectroscopy and fluorescence spectroscopy.

4. The device of claim 3, wherein said additional assessment further comprises at least one fluorescence imaging mode.

5. The device of claim 1, further comprising means for performing an action based on said additional assessment.

6. The device of claim 5, wherein said action comprises at least one of an audible alert and a visible alert.

7. The device of claim 6, further comprising means for manually changing a visual output mode after said alert.

8. The device of claim 7, wherein said means for manually changing further comprises at least one of means for displaying fluorescence images, means for displaying spectroscopic data, means for displaying composite images, means for highlighting said visual output mode, means for delineating regions of said visual output mode and means for overlaying said visual output mode.

9. The device of claim 7, further comprising means for automatically changing a visual output mode after said alert.

10. The device of claim 9, wherein said means for automatically changing further comprises at least one of means for displaying fluorescence images, means for displaying spectroscopic data, means for displaying composite images, means for highlighting said visual output mode, means for delineating regions of said visual output mode and means for overlaying said visual output mode.

11. The device of claim 1, further comprising means to calculate a quantitative score based on said additional assessment.

12. The device of claim 11, further comprising means to compare said quantitative score to a benchmark score.

13. The device of claim 11, further comprising means to display said quantitative score and said benchmark score.

14. The device of claim 1, further comprising means for performing an action based on said additional assessment and on prior information relating to the target.

15. The device of claim 14, wherein said action comprises at least one of an audible alert and a visible alert.

16. The device of claim 15, further comprising means for manually changing a visual output mode after said alert.

17. The device of claim 16, wherein said means for manually changing further comprises at least one of means for displaying fluorescence images, means for displaying spectroscopic data, means for displaying composite images, means for highlighting said visual output mode, means for delineating regions of said visual output mode and means for overlaying said visual output mode.

18. The device of claim 15, further comprising means for automatically changing a visual output mode after said alert.

19. The device of claim 18, wherein said means for automatically changing further comprises at least one of means for displaying fluorescence images, means for displaying spectroscopic data, means for displaying composite images, means for highlighting said visual output mode, means for delineating regions of said visual output mode and means for overlaying said visual output mode.

20. The device of claim 14, further comprising means to calculate a quantitative score based on said additional assessment and on prior information relating to the target.

21. The device of claim 20, further comprising means to compare said quantitative score to a benchmark score.

22. The device of claim 20, further comprising means to display said quantitative score and said benchmark score.

23. The device of claim 1, further comprising means for performing an action based on said additional assessment and an analysis from a plug-in analyzer.

24. The device of claim 23, wherein said plug-in analyzer comprises at least one of a Raman probe, a fluorescence excitation-emission matrix spectroscopy probe, an optical coherence tomography probe, and a confocal microscopy probe.

25. The device of claim 23, wherein said action comprises at least one of an audible alert and a visible alert.

26. The device of claim 25, further comprising means for manually changing a visual output mode after said alert.

27. The device of claim 26, wherein said means for manually changing further comprises at least one of means for displaying fluorescence images, means for displaying spectroscopic data, means for displaying composite images, means for highlighting said visual output mode, means for delineating regions of said visual output mode and means for overlaying said visual output mode.

28. The device of claim 25, further comprising means for automatically changing a visual output mode after said alert.

29. The device of claim 28, wherein said means for automatically changing further comprises at least one of means for displaying fluorescence images, means for displaying spectroscopic data, means for displaying composite images, means for highlighting said visual output mode, means for delineating regions of said visual output mode and means for overlaying said visual output mode.

30. The device of claim 1, further comprising means to calculate a quantitative score based on said additional assessment and an analysis from a plug-in analyzer.

31. The device of claim 30, further comprising means to compare said quantitative score to a benchmark score.

32. The device of claim 30, further comprising means to display said quantitative score and said benchmark score.

33. The device of claim 1, further comprising an endoscopy positioning system.

34. An automated method for imaging and diagnosing a target, comprising:
illuminating the target with white light; and
assessing the target as a background task.

35. The method of claim 34, wherein said assessing step comprises at least fluorescence imaging.

36. The method of claim 34, wherein assessing step comprises at least one of reflectance spectroscopy and fluorescence spectroscopy.

37. The method of claim 36, wherein said assessing step further comprises at least fluorescence imaging.

38. The method of claim 34, further comprising performing an action based on a result of said assessing step.

39. The method of claim 38, wherein said action comprises at least one of an audible alert and a visible alert.

40. The method of claim 39, further comprising manually changing a visual output mode after said alert.

41. The method of claim 40, wherein said manually changing step further comprises at least one of displaying fluorescence images, displaying spectroscopic data, displaying composite images, highlighting said visual output mode, delineating regions of said visual output mode and overlaying said visual output mode.

42. The method of claim 39, further comprising automatically changing a visual output mode after said alert.

43. The method of claim 42, wherein said means for automatically changing step further comprises at least one of displaying fluorescence images, displaying spectroscopic data, displaying composite images, highlighting said visual output mode, delineating regions of said visual output mode and overlaying said visual output mode.

44. The method of claim 34, further comprising calculating a quantitative score based on said additional assessment.

45. The method of claim 44, further comprising comparing said quantitative score to a benchmark score.

46. The method of claim 44, further comprising displaying said quantitative score and said benchmark score.

47. The method of claim 34, further comprising performing an action based on said additional assessment and on prior information relating to the target.

48. The method of claim 47, wherein said action comprises at least one of an audible alert and a visible alert.

49. The method of claim 48, further comprising manually changing a visual output mode after said alert.

50. The method of claim 49, wherein said manually changing step further comprises at least one of displaying fluorescence images, displaying spectroscopic data, displaying composite images, highlighting said visual output mode, delineating regions of said visual output mode and overlaying said visual output mode.

51. The method of claim 48, further comprising automatically changing a visual output mode after said alert.

52. The method of claim 51, wherein said automatically changing step further comprises at least one of displaying fluorescence images, displaying spectroscopic data, displaying composite images, highlighting said visual output mode, delineating regions of said visual output mode and overlaying said visual output mode.

53. The method of claim 47, further comprising calculating a quantitative score based on said additional assessment and on prior information relating to the target.

54. The method of claim 53, further comprising comparing said quantitative score to a benchmark score.

55. The method of claim 53, further comprising displaying said quantitative score and said benchmark score.

56. The method of claim 34, further comprising performing an action based on said additional assessment and an analysis from a plug-in analyzer.

57. The method of claim 56, wherein said plug-in analyzer comprises at least one of a Raman probe, a fluorescence excitation-emission matrix spectroscopy probe, an optical coherence tomography probe, and a confocal microscopy probe.

58. The method of claim 56, wherein said action comprises at least one of an audible alert and a visible alert.

59. The method of claim 58, further comprising manually changing a visual output mode after said alert.

60. The method of claim 59, wherein said manually changing step further comprises at least one of displaying fluorescence images, displaying spectroscopic data, displaying composite images, highlighting said visual output mode, delineating regions of said visual output mode and overlaying said visual output mode.

61. The method of claim 58, further comprising automatically changing a visual output mode after said alert.

62. The method of claim 61, wherein said automatically changing step further comprises at least one of displaying fluorescence images, displaying spectroscopic data, displaying composite images, highlighting said visual output mode, delineating regions of said visual output mode and overlaying said visual output mode.

63. The method of claim 34, further comprising calculating a quantitative score based on said additional assessment and an analysis from a plug-in analyzer.

64. The method of claim 63, further comprising comparing said quantitative score to a benchmark score.

65. The method of claim 63, further comprising displaying said quantitative score and said benchmark score.

66. The method of claim 34, further comprising using an endoscopy positioning system.
